(19) United States
(12) Patent Application Publication
(10) Pub. No.: US 2025/0320473 A1
(43) Pub. Date: Oct. 16, 2025

(54) CHIMERA DNA POLYMERASE AND PREPARATION METHOD THEREFOR

(71) Applicant: WUHAN ABCLONAL BIOTECHNOLOGY CO., LTD., Wuhan (CN)

(72) Inventors: Yanye Feng, Wuhan (CN); Zhi Chai, Wuhan (CN); Jie Feng, Wuhan (CN); Shaohui Liu, Wuhan (CN); Dapeng Sun, Wuhan (CN); Xuhui Lai, Wuhan (CN)

(73) Assignee: WUHAN ABCLONAL BIOTECHNOLOGY CO., LTD., Wuhan, HB (CN)

(21) Appl. No.: 18/694,872

(22) PCT Filed: Feb. 16, 2022
(86) PCT No.: PCT/CN2022/071597

§ 371 Date: Mar. 22, 2024

(30) Foreign Application Priority Data
Sep. 23, 2021 (CN) 202111114932.5

(51) Int. Cl. C12N 9/12; C12P 19/34

(52) U.S. Cl. CPC C12N 9/1252; C12P 19/34; C07K 2319/80; C12Y 207/07007

(57) ABSTRACT

A chimera DNA polymerase and a preparation method therefor. The chimera DNA polymerase contains 2-8 (for example 3) domains or segments derived from different polymerases. The chimera DNA polymerase has improved properties such as a better extension characteristic, a better DNA binding characteristic, a better corrective activity, a better fidelity, a higher amplification speed, a better tolerance to an inhibitor, and a higher long fragment amplification capability.

### CROSS-REFERENCE TO RELATED APPLICATIONS

This application claims priority to CN patent application Ser. No. 202111114932.5, filed on Sep. 23, 2021, and entitled “CHIMERA DNA POLYMERASE AND PREPARATION METHOD THEREFOR”, which is incorporated herein by reference in its entirety.

### REFERENCE TO SEQUENCE LISTING

A Sequence Listing is provided as a file titled PD210083PCT-US_sequence_list.txt created Apr. 25, 2025, which is approximately 1,819 KB in size, and which includes an English language translation of the Sequence Listing originally submitted with the present application. The material in this file is incorporated herein by reference in its entirety.

### TECHNICAL FIELD

This application relates to the field of enzyme engineering. Specifically, this application relates to a chimera polymerase and a preparation method and use therefor.

### BACKGROUND

A polymerase is a collective name of a type of enzymes that specifically biologically catalyze syntheses of deoxyribonucleic acid (DNA) and ribonucleic acid (RNA). In 1957, American scientist Arthur Kornberg discovered a DNA polymerase in Escherichia coli for the first time. The enzyme was named DNA polymerase I. In 1970, German scientist Rolf Knippers discovered DNA polymerase II. Subsequently, DNA polymerase III was discovered.

As one of important factors in the polymerase chain reaction (PCR), DNA polymerase has a crucial function in the PCR process. A PCR technology is a technology of a thermostable DNA polymerase in a sense. Thermostable DNA polymerases that have been discovered all belong to family A or family B. DNA polymerases belonging to the family A are all derived from eubacteria, for example, Taq, Tth, Tca (T. caldophilus), Tfl, and Tfl derived from Thermus genus, and Bst derived from Bacillus genus; and thermostable DNA polymerases belonging to the family B are all derived from archaea, for example, Tli derived from Thermococcus genus, and Pfu and KOD derived from Pyrococcus genus, and the like.

Since the advent of the PCR technology, people have been continuously looking for DNA polymerases with good enzymatic properties and high fidelity for use in the PCR. After Taq DNA polymerase, thermostable DNA polymerases with proofreading functions such as Deep Vent, Pfu, Tgo, and KOD were successively discovered.

As a technology for amplifying specific DNA fragments in vitro rapidly, the polymerase chain reaction (PCR) is a DNA polymerase-catalyzed reaction for amplifying DNA fragments defined by a pair of oligonucleotide primers in a reaction mixture consisting of DNA templates, primers, dNTPs, appropriate buffers, and the like. In this process, the DNA polymerase has a crucial function. Development and utilization of enzymes is one of important contents of modern biotechnologies. Using the technologies to modify and design enzyme genes is one of important means of biological enzyme engineering.

### SUMMARY

This application provides a chimera DNA polymerase with high fidelity. The chimera DNA

polymerase in this application may further have improved properties such as processivity, DNA binding activity, proofreading activity, fidelity, amplification speed, the tolerance to inhibitors, and long fragment amplification capability.

### DNA Polymerase

The chimera DNA polymerase in this application may contain 2-8 (for example, 3) domains or segments derived from different polymerases. The domains or segments include, but are not limited to, an exonuclease domain (generally referring to an N-terminal region), a thumb domain, a palm structure, and a finger domain. The domains or segments can be derived from different DNA polymerases, including but are not limited to: Pfu polymerase, KOD polymerase, 9N polymerase, T4 polymerase, and phi29 polymerase. The polymerases can be derived from various thermophilic bacteria, including, but not limited to, Thermotoga sp, Thermococcus profundus, Thermococcus gammatolerans, Thermococcus radiotolerans, Pyrococuus sp.NA2, Thermococcus celericrescens, Pyrococcus glycovorans, and Pyrococcus furiosus. An identity between the chimera DNA polymerases in this application can be more than 80%.

In some embodiments, a chimera DNA polymerase with DNA replication activity is provided, including:
a first domain, encoded by a nucleotide sequence selected from nucleotide sequences denoted as SEQ ID NOs: 576 to 583 or a nucleotide sequence that shares a sequence identity of at least 70%, 75%, 80%, 85%, 90%, 95%, 97%, 98% or 99% with one of the nucleotide sequences denoted as SEQ ID NOs: 576 to 583;
a second domain, encoded by a nucleotide sequence selected from nucleotide sequences denoted as SEQ ID NOs: 584 to 591 or a nucleotide sequence that shares a sequence identity of at least 70%, 75%, 80%, 85%, 90%, 95%, 97%, 98% or 99% with one of the nucleotide sequences denoted as SEQ ID NOs: 584 to 591; and
a third domain, encoded by a nucleotide sequence selected from nucleotide sequences denoted as SEQ ID NOs: 592 to 599 or a nucleotide sequence that shares a sequence identity of at least 70%, 75%, 80%, 85%, 90%, 95%, 97%, 98% or 99% with one of the nucleotide sequences denoted as SEQ ID NOs: 592 to 599.

More specifically, nucleotide sequences denoted as SEQ ID NOs: 576 to 599 are respectively derived from eight source species: Thermococcus profundus (Thermococcus profundus), Thermococcus gammatolerans (Thermococcus gammatolerans), Thermococcus radiotolerans (Thermococcus radiotolerans), Pyrococcus sp. NA2 (Pyrococcus sp. NA2), Thermococcus celericrescens (Thermococcus celericrescens), Pyrococcus glycovorans (Pyrococcus glycovorans), and Pyrococcus furiosus (Pyrococcus furiosus). The first domain encoded by a nucleotide sequence selected from the nucleotide sequences denoted as SEQ ID NOs: 576 to 583 is the N-terminal domain, which is mainly involved in a proofreading or exonucleation function of 3′-5′ exonuclease activity; and the second domain encoded by a nucleotide sequence selected from the nucleotide sequences denoted as SEQ ID NOs: 584 to 591 is the finger and palm domains. The finger domain or the palm domain is mainly responsible for binding and incorporation of dNTPs and is an active center of the enzyme. The third domain encoded by a nucleotide sequence selected from the nucleotide sequences denoted as SEQ ID NOs: 592 to 599 is the thumb domain, which is mainly related to a capability of processivity. There is no absolute cleavage among the three regions. Conservative cleavage and combination are carried out based on structure and sequence characteristics to construct diversity of an enzyme library.

In some more specific embodiments, a combined polymerase with the first domain of SEQ ID NO: 583 or 581, the second domain of SEQ ID NO: 586 or 591, and the third domain of SEQ ID NO: 596 or 598 has a good processivity; and a combined polymerase with the first domain of SEQ ID NO: 578 or 582, the second domain of SEQ ID NO: 586 or 590, and the third domain of SEQ ID NO: 592, 593, or 594 has a greater extension rate.

In some embodiments, the chimera DNA polymerase in this application includes a first domain encoded by a nucleotide sequence selected from nucleotide sequences denoted as SEQ ID NOs: 576 to 583, a second domain encoded by a nucleotide sequence selected from nucleotide sequences denoted as SEQ ID NOs: 584 to 591, and the third domain encoded by a nucleotide sequence selected from nucleotide sequences denoted as SEQ ID NOs: 592 to 599, or consists of the foregoing first domain, second domain, and third domain.

In some other embodiments, the chimera DNA polymerase including the foregoing first domain, second domain and third domain in this application further contains or has one or more amino acid substitutions. For example, the amino acid substitution may be selected from one or more amino acid substitutions corresponding to amino acids at the following positions: 5, 6, 11, 15, 16, 18, 22, 24, 25, 28, 30, 33, 35, 36, 38, 43, 47, 49, 50, 51, 52, 54, 56, 57, 61, 62, 64, 65, 66, 67, 68, 72, 73, 80, 81, 84, 88, 89, 90, 94, 96, 99, 100, 102, 104, 107, 110, 126, 127, 132, 136, 137, 138, 139, 140, 153, 154, 158, 165, 166, 167, 169, 176, 180, 182, 183, 185, 186, 188, 189, 193, 194, 195, 196, 197, 198, 199, 206, 210, 213, 216, 217, 220, 223, 226, 228, 230, 231, 232, 233, 236, 238, 241, 244, 247, 248, 251, 252, 261, 262, 265, 268, 282, 285, 286, 292, 293, 296, 297, 301, 302, 303, 304, 310, 318, 320, 324, 327, 331, 334, 337, 340, 341, 356, 367, 373, 374, 375, 377, 378, 379, 383, 384, 386, 395, 399, 400, 401, 403, 406, 407, 408, 409, 410, 424, 426, 430, 434, 437, 439, 441, 446, 447, 455, 456, 459, 463, 466, 467, 470, 471, 472, 475, 477, 478, 479, 485, 494, 499, 502, 508, 520, 524, 525, 526, 527, 529, 532, 533, 540, 545, 546, 552, 553, 554, 556, 557, 559, 560, 562, 565, 566, 570, 575, 585, 588, 597, 604, 605, 626, 631, 633, 634, 636, 642, 646, 652, 653, 656, 658, 662, 664, 670, 672, 673, 677, 683, 690, 692, 694, 695, 698, 701, 703, 706, 708, 710, 712, 713, 717, 718, 719, 721, 723, 724, 727, 743, 747, 752, 753, 755, 758, 762, 764, 767, 768, 771, 772, 774, and 775, where the positions are defined with reference to SEQ ID NO: 575.

For example, the amino acid substitution can be selected from one or more of the following:
V5T/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V1071, F110Y, L1261, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, 1197V, 1198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V2521, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, I434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, I478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, 1656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K, where the positions are defined with reference to SEQ ID NO: 575.

The polypeptide denoted as SEQ ID NO: 575 is derived from Pyrococcus furiosus, and contains three domains having the following sequences:

Domain 1:
atgattttagatgtggattacataactgaagaaggaaaacctgttattag
gctattcaaaaaagagaacggaaaatttaagatagagcatgatagaactt
ttagaccatacatttacgctcttctcagggatgattcaaagattgaagaa
gttaagaaaataacgggggaaaggcatggaaagattgtgagaattgttga
tgtagagaaggttgagaaaaagtttctcggcaagcctattaccgtgtgga
aactttatttggaacatccccaagatgttcccactattagagaaaaagtt
agagaacatccagcagttgtggacatcttcgaatacgatattccatttgc
aaagagatacctcatcgacaaaggcctaataccaatggagggggaagaag
agctaaagattcttgccttcgatatagaaaccctctatcacgaaggagaa
gagtttggaaaaggcccaattataatgattagttatgcagatgaaaatga
agcaaaggtgattacttggaaaaacatagatcttccatacgttgaggttg
tatcaagcgagagagagatgataaagagatttctcaggattatcagggag
aaggatcctgacattatagttacttataatggagactcattcgacttccc
atatttagcgaaaagggcagaaaaacttgggattaaattaaccattggaa
gagatggaagcgagcccaagatgcagagaataggcgatatgacggctgta
gaagtcaagggaagaatacatttcgacttgtatcatgtaataacaaggac
aataaatctcccaacatacacactagaggctgtatatgaagcaatttttg
gaaagccaaaggagaaggtatacgccgacgagatagcaaaagcctgggaa
agtggagagaaccttgagagagttgccaaatactcgatggaagatgcaaa
ggcaacttatgaactcgggaaagaattccttccaatggaaattcagcttt
caagattagttggacaacctttatgggatgtttcaaggtcaagcacaggg
aaccttgtagagtggttcttacttaggaaagcctacgaaagaaacgaa
Domain 2:
gtagctccaaacaagccaagtgaagaggagtatcaaagaaggctcaggga
gagctacacaggtggattcgttaaagagccagaaaaggggttgtgggaaa
acatagtatacctagattttagagccctatatccctcgattataattacc
cacaatgtttctcccgatactctaaatcttgagggatgcaagaactatga
tatcgctcctcaagtaggccacaagttctgcaaggacatccctggtttta
taccaagtctcttgggacatttgttagaggaaagacaaaagattaagaca
aaaatgaaggaaactcaagatcctatagaaaaaatactccttgactatag
acaaaaagcgataaaactcttagcaaattctttctacggatattatggct
atgcaaaagcaagatggtactgtaaggagtgtgctgagagcgttactgcc
tggggaagaaagtacatcgagttagtatggaaggagctcgaagaaaagtt
tggatttaaagtcctctacattgacactgatggtctctatgcaactatcc
caggaggagaaagtgaggaaataaagaaaaaggctctagaatttgtaaaa
tacataaattcaaagctccctggactgctagagcttgaatatgaagggtt
ttataagaggggattcttcgttacgaagaagaggtatgcagtaatagatg
aagaaggaaaagtcattactcgtggtttagagatagttaggagagattgg
agtgaaattgcaaaagaaactcaa
Domain 3:
gctagagttttggagacaatactaaaacacggagatgttgaagaagctgt
gagaatagtaaaagaagtaatacaaaagcttgccaattatgaaattccac
cagagaagctcgcaatatatgagcagataacaagaccattacatgagtat
aaggcgataggtcctcacgtagctgttgcaaagaaactagctgctaaagg
agttaaaataaagccaggaatggtaattggatacatagtacttagaggcg
atggtccaattagcaatagggcaattctagctgaggaatacgatcccaaa
aagcacaagtatgacgcagaatattacattgagaaccaggttcttccagc
ggtacttaggatattggagggatttggatacagaaaggaagacctcagat
accaaaagacaagacaagtcggcctaacttcctggcttaacattaaaaaa
tcctag

In some embodiments, the chimera DNA polymerase in this application has improved properties such as better Mg²⁺ tolerance, better SDS tolerance, better TE tolerance, and a higher long fragment amplification capability.

In some embodiments, the amino acid substitution may be selected from one or more amino acid substitutions corresponding to amino acids at the following positions: 210, 213, 377, 378, 407, 408, 409, 410, 474, and 501. The inventor of this application has discovered that amino acids at positions 408, 409 and/or 410 are related to a binding capability of dNTPs and all belong to the active center, which directly affects amplification efficiency and yield of the polymerase; amino acids at positions 210 and/or 213 are related to tolerance to an inhibitor, for example, when amino acids at positions 210 and 213 are D, the amino acids significantly increase a range of the tolerance to the inhibitor; the amino acids at the positions 210 and/or 213 are directly related to exonuclease activity because mutations at such sites are directly related to fidelity and proofreading activity of the polymerase; amino acids at positions 501, 474, and/or 377 are related to amplification efficiency of the polymerase, and therefore, mutations at such sites can improve yield of fragments of amplification targets; an amino acid at position 378 is directly related to tolerance to SDS; and an amino acid at position 407 is directly related to tolerance to Mg and TE.

This application further provides a DNA polymerase mutant with DNA replication activity, including an amino acid sequence, where when compared with a reference polypeptide denoted as SEQ ID NO: 575, the amino acid sequence includes one or more amino acid substitutions corresponding to amino acids at the following positions: 5, 6, 11, 15, 16, 18, 22, 24, 25, 28, 30, 33, 35, 36, 38, 43, 47, 49, 50, 51, 52, 54, 56, 57, 61, 62, 64, 65, 66, 67, 68, 72, 73, 80, 81, 84, 88, 89, 90, 94, 96, 99, 100, 102, 104, 107, 110, 126, 127, 132, 136, 137, 138, 139, 140, 153, 154, 158, 165, 166, 167, 169, 176, 180, 182, 183, 185, 186, 188, 189, 193, 194, 195, 196, 197, 198, 199, 206, 210, 213, 216, 217, 220, 223, 226, 228, 230, 231, 232, 233, 236, 238, 241, 244, 247, 248, 251, 252, 261, 262, 265, 268, 282, 285, 286, 292, 293, 296, 297, 301, 302, 303, 304, 310, 318, 320, 324, 327, 331, 334, 337, 340, 341, 356, 367, 373, 374, 375, 377, 378, 379, 383, 384, 386, 395, 399, 400, 401, 403, 406, 407, 408, 409, 410, 424, 426, 430, 434, 437, 439, 441, 446, 447, 455, 456, 459, 463, 466, 467, 470, 471, 472, 475, 477, 478, 479, 485, 494, 499, 502, 508, 520, 524, 525, 526, 527, 529, 532, 533, 540, 545, 546, 552, 553, 554, 556, 557, 559, 560, 562, 565, 566, 570, 575, 585, 588, 597, 604, 605, 626, 631, 633, 634, 636, 642, 646, 652, 653, 656, 658, 662, 664, 670, 672, 673, 677, 683, 690, 692, 694, 695, 698, 701, 703, 706, 708, 710, 712, 713, 717, 718, 719, 721, 723, 724, 727, 743, 747, 752, 753, 755, 758, 762, 764, 767, 768, 771, 772, 774, and 775, where the positions are defined with reference to SEQ ID NO: 575. In some embodiments, the amino acid substitution is selected from one or more of the following:
V5T/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V107I, F110Y, L1261, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, I198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V2521, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, I434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, 1478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, 1605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K.

In some embodiments, the DNA polymerase mutant shares a sequence identity of at least 70%, 75%, 80%, 85%, 90%, 95%, 97%, 98% or 99% reference polypeptide denoted as SEQ ID NO: 575.

In some embodiments, a DNA polymerase mutant in this application has improved properties such as better Mg²⁺ tolerance, better SDS tolerance, better TE tolerance, and a higher long fragment amplification capability.

In some embodiments, an amino acid sequence of the DNA polymerase mutant includes one or more amino acid substitutions corresponding to amino acids at the following positions: 210, 213, 377, 378, 407, 408, 409, 410, 474, and 501.

In some embodiments, the DNA polymerase in this application includes an amino acid sequence, sharing a sequence identity of at least 70%, 75%, 80%, 85%, 90%, 95%, 97%, 98% or 99% with any one of the amino acid sequences denoted as SEQ ID NOs: 1 to 574. In some embodiments, the DNA polymerase in this application includes any one of the amino acid sequences denoted as SEQ ID NOs: 1 to 574. Examples are as follows:

A polymerase with SEQ ID NO: 564 consists of the first domain with SEQ ID NO: 583, the second domain with SEQ ID NO: 586, and the third domain with SEQ ID NO: 596, and includes V5A, D6N, E11D, V15I, L18I, K25E, I28V, H30Y, T33N, R43K, K47Q, E49D, G56A, E72K, K78R, T81E, L86F, T95A, E98D, V100I, E132D, I137L, G153A, E167G, N175K, I176V, R196K, I197V, I205V, V2071, F216L, A220V, T231P, I232L, I244L, V252I, T265R, D293H, K297E, S301T, E303K, N304G, A318V, K324R, L327F, 1331A, F356Y, and V367I.

A polymerase with SEQ ID NO: 561 consists of the first domain with SEQ ID NO: 583, the second domain with SEQ ID NO: 586, and the third domain with SEQ ID NO: 598, and includes V5A, D6N, E11D, V15I, L18I, K25E, I28V, H30Y, T33N, R43K, K47Q, E49D, G56A, E72K, K78R, T81E, L86F, T95A, E98D, V100I, E132D, G153A, E167G, N175K, I176V, R196K, I197V, I205V, V207I, F216L, A220V, T231P, I232L, I244L, V252I, T265R, D293H, K297E, S301T, E303K, N304G, A318V, K324R, L327F, 1331A, F356Y, and V367I.

A polymerase with SEQ ID NO: 287 consists of the first domain with SEQ ID NO: 519, the second domain with SEQ ID NO: 584, and the third domain with SEQ ID NO: 595, and includes V5A, E11D, V151, K25R, I28V, H30Y, T33N, R43K, K47A, E49D, E50D, K52R, G56S, E57K, I62V, I65V, V66I, E72K, K78R, T81E, L86F, T95A, 196M, E98D, V100I, V107I, E132N, K136T, F140V, E167G, N175K, S185A, S186N, F194L, L195I, R196K, I197V, I205V, V207I, S213N, P217A, A220L, I228M, T231P, I232L, G236N, I244L, M247S, T248L, V252I, Y261F, H262P, T265R, P286Q, A292P, D293H, K297E, S301T, E303K, N304G, A318V, L327F, I331A, S334A, F356Y, and V367L.

A polymerase with SEQ ID NO: 503 consists of the first domain with SEQ ID NO: 581, the second domain with SEQ ID NO: 590, and the third domain with SEQ ID NO: 594, and includes E10D, V14I, L181, I28V, H30Y, T33N, R43K, K47Q, K52R, G56A, V66I, K78R, K83R, L86F, T95A, E98D, P104S, V107I, E132D, N175K, R196K, 1205V, V207I, F216L, A220V, I244L, V252I, K297E, K324R, and V367L.

A polymerase with SEQ ID NO: 532 consists of the first domain with SEQ ID NO: 583, the second domain with SEQ ID NO: 588, and the third domain with SEQ ID NO: 596, and includes V5A, D6N, E11D, V15I, L18I, K25E, I28V, H30Y, T33N, R43K, K47Q, E49D, G56A, E72K, K78R, T81E, L86F, T95A, E98D, V100I, E132D, G153A, E167G, N175K, I176V, R196K, I197V, I205V, V207I, F216L, A220V, T231P, I232L, I244L, V252I, T265R, D293H, K297E, S301T, E303K, N304G, A318V, K324R, L327F, I331A, F356Y, and V367L.

A polymerase with SEQ ID NO: 78 consists of the first domain with SEQ ID NO: 578, the second domain with SEQ ID NO: 586, and the third domain with SEQ ID NO: 598, and includes V5T, E11N, L18V, K24E, H30Y, R35E, I38F, R43K, K47A, E50D, I54V, G56A, K61T, I65V, V66K, D67R, V68A, E72Q, K73R, K78R, T81E, L86F, E87T, T95A, E98D, V100I, E102A, V1071, F110Y, E132D, K136T, K154T, I158L, E165G, E166S, K169R, N175K, E182D, S186T, R188K, I198V, R199K, I205V, I206L, V2071, S213N, A220K, A223C, L230F, I232L, M301I, I244M, M247R, T248F, H262P, T265R, I282V, D293E, K297Q, N304G, K310R, A318V, K324R, L327F, I331A, V337I, P340S, and V367I.

A polymerase with SEQ ID NO: 406 consists of the first domain with SEQ ID NO: 580, the second domain with SEQ ID NO: 591, and the third domain with SEQ ID NO: 596, and includes E11D, I15V, I16V, L18I, K25E, H30Y, T33N, R35E, R43K, K47A, G56A, 162V, I65V, V66K, D67R, V68A, E72K, K78R, T81E, L86F, T95A, E98D, V100I, F110Y, E132D, I158L, K169R, N175K, S186T, 1197V, R199K, I205V, I206L, V2071, S213N, P217A, A220K, A223C, L230F, I232L, M241I, I244M, M247R, T248F, E251D, H262P, T265R, D293E, K297E, S301T, N304G, K310R, A318V, K324R, L327F, I331A, V337I, and V367L.

A polymerase with SEQ ID NO: 403 consists of the first domain with SEQ ID NO: 580, the second domain with SEQ ID NO: 591, and the third domain with SEQ ID NO: 598, and includes E11D, I15V, I16V, L18I, K25E, H30Y, T33N, R35E, R43K, K47A, G56A, I62V, I65V, V66K, D67R, V68A, E72K, K78R, T81E, L86F, T95A, E98D, V100I, F110Y, E132D, I158L, K169R, N175K, S186T, 1197V, R199K, I205V, I206L, V2071, S213N, P217A, A220K, A223C, L230F, I232L, M241I, 1244M, M247R, T248F, E251D, H262P, T265R, D293E, K297E, S301T, N304G, K310R, A318V, K324R, L327F, I331A, V337I, and V367L.

This application also relates to biologically active fragments of the DNA polymerase in this application, and such fragments are considered to be included in terms “DNA polymerase in this application”, “chimera DNA polymerase in this application”, and “DNA polymerase mutant in this application”. The biologically active fragment of the DNA polymerase in this application includes fewer amino acids than a full-length protein, but exhibits at least one biological activity of the corresponding full-length protein. Generally, the biologically active fragment includes at least one domain or motif or segment of the DNA polymerase protein in this application. A biologically active fragment that lacks a local region of a protein can be prepared through recombination techniques, and the fragment is evaluated for one or more biological activities possessed by a full-length form of the DNA polymerase in this application.

The term “DNA polymerase” used in this application refers to an enzyme for replicating DNA, and replicates DNA from the 5′-end to the 3′-end by using DNA as a replication template. The DNA polymerase has an activity of catalyzing DNA syntheses in the presence of templates, primers, dNTPs, and the like and optionally, has auxiliary activities.

The term “amino acid” used in this application is a compound obtained by substituting a hydrogen atom on a carbon atom of carboxylic acid with an amino group. The amino acid molecule contains two functional groups: the amino group and carboxyl group. Similar to hydroxy acids, amino acids can be divided into α-amino acid, β-amino acid, γ-amino acid, . . . , w-amino acid based on different positions of amino groups on carbon chains, but amino acids obtained after protein hydrolysis are all α-amino acids, belong to only two dozen categories, and are basic units for forming proteins.

The term “PCR” or “polymerase chain reaction” used in this application is a molecular biology technology for amplifying specific DNA fragments, and can be regarded as special DNA replication in vitro. DNA is denatured into single strands at higher temperature of 95° C. in vitro. At lower temperature (usually around 60° C.), the primers and the single strands are combined according to a complementary base pairing rule, then the temperature is adjusted to optimal reaction temperature (around 72° C.) of the DNA polymerase, and the DNA polymerase synthesizes complementary strands along a direction from phosphate to pentose (5′-3′). A PCR machine manufactured based on polymerase is actually a temperature control device that can control the denaturation temperature, renaturation temperature, and extension temperature well.

There are five main types of substances participating in the PCR reaction, namely, primers, enzymes, dNTPs, templates and Mg²⁺, which can be called reaction elements. The primers are crucial to specific PCR reactions. Specificity of a PCR product depends on a degree of complementarity between the primers and the template DNA. Mg²⁺ has significant impact on specificity and yield of PCR amplification. In common PCR reactions, when concentrations of various dNTPs are 200 umol/L, an appropriate concentration of Mg²⁺ is 1.5 mmol/L to 2.0 mmol/L. If the concentration of Mg²⁺ is too high, reaction specificity is reduced and non-specific amplification occurs. If the concentration is too low, activity of the DNA polymerase is reduced and the reaction product is reduced.

The term “domain” used in this application refers to any structural fragment or specifically active region of the polymerase, for example, a DNA binding region, a nucleotide polymerization region, a dNTP binding region, a strand displacement binding region, or a region with proofreading activity.

The term “inhibitor tolerance” used in this application refers to a capability of the DNA polymerase to substantially maintain its enzymatic activity in the presence of substances that have an adverse effect on the PCR, including but not limited to Mg²⁺ tolerance, SDS tolerance, and TE tolerance. The tolerance to the inhibitor can be measured via the maximum inhibitor concentration at which the DNA polymerase still substantially has activity. In this application, the “Mg²⁺ tolerance” may refer to a capability of substantially maintaining activity of the DNA polymerase in the presence of Mg²⁺ at a concentration of more than 2 mM, 4 mM, 6 mM, 8 mM, or 10 mM. In this application, the “SDS tolerance” may refer to a capability of substantially maintaining the activity of the DNA polymerase in the presence of 0.00125% SDS, 0.0025% SDS, 0.005% SDS, 0.01% SDS, or 0.02% SDS. In this application, the “TE tolerance” may refer to a capability of substantially maintaining the activity of the DNA polymerase in the presence of 0.03125× TE, 0.0625× TE, 0.125× TE, 0.25× TE, 0.5× TE, or 1× TE. The term “substantially” used herein means that the DNA polymerase maintains 10%, 20%, 30%, 40%, 50%, 60%, 70%, 75%, 80%, 85%, 90%, 95%, 98%, 99% or above of the DNA polymerase activity in any or a specific target assay in vivo or in vitro. The target assay can be a semi-quantitative or quantitative PCR amplification experiment. Alternatively, the target assay may be, for example, a DNA binding assay, a nucleotide polymerization assay, a primer extension assay, a strand displacement assay, a reverse transcriptase assay, a proofreading assay, an accuracy assay, a thermal stability assay, or an ion stability assay.

The term “long fragment amplification capability” used in this application refers to a capability of the DNA polymerase to generate long fragments through the PCR reactions. In this application, the “long fragment amplification capability” may refer to the capability of amplifying continuous DNA fragments greater than 1 kb, 2 kb, 3 kb, 4 kb, 5 kb, 6 kb, 7 kb, 8 kb, 9 kb, or 10 kb.

The term “substitution” or “amino acid substitution” used herein refers to replacement of at least one amino acid residue in a specific amino acid sequence by another different amino acid residue. The representation of substitution is well known in the art. For example, T5V/A refers to substitution of T in the 5^{th} site with V or A, and D6N refers to substitution of D in the 6^{th} site with N. In some embodiments, the amino acid substitution is a conservative substitution. The “conserved substitution” means that one amino acid is replaced with another amino acid that has a common property. A method of functionally defining the common property of individual amino acids is to analyze a normalized frequency of amino acid changes between corresponding proteins in homologous organisms (Schulz (1979) Principles of Protein Structure, Springer-Verlag). Based on such an analysis, a family of amino acids can be determined, amino acids within the family are preferentially interchanged with each other, and have the most similar effects on the overall structure of the protein (Schulz (1979) ibid.). Examples of groups of amino acids defined in this way include: “charged/polar family”, including Glu, Asp, Asn, Gln, Lys, Arg, and His; “aromatic or ring family”, including Pro, Phe, Tyr and Trp; and “aliphatic family”, including Gly, Ala, Val, Leu, Ile, Met, Ser, Thr, and Cys. Within each family, subfamilies can also be determined. For example, the family of charged/polar amino acids can be further divided into subfamilies, the subfamilies include: a “positively charged subfamily” including Lys, Arg, and His; a “negatively charged subgroup” including Glu and Asp; and a “polar subfamily” including Asn and Gln. For another example, the aromatic or cyclopedic family can be further subdivided into subfamilies, including: “nitrogen ring subfamily” including Pro, His, and Trp; and the “phenyl subfamily”, including Phe and Tyr. For another example, the aliphatic group can be further divided into subfamilies, including: “large aliphatic non-polar subfamily”, including Val, Leu, and Ile; “aliphatic micropolar subfamily”, including Met, Ser, Thr, and Cys; and the “little residue subfamily”, including Gly and Ala. An example of a conserved mutation includes an amino acid substitution of an amino acid within the above subfamily, including but not limited to: a substitution of Arg with Lys or vice versa, to maintain a positive charge; a substitution of Asp with Glu or vice versa, which can maintain a negative charge; a substitution of Thr with Ser or vice versa, which can maintain free-OH; or a substitution of Asn with Gln or vice versa, which can maintain free —NH₂. A “conserved variant” is a peptide that contains one or more amino acids that have been replaced with an amino acid having a common property (belonging to, for example, the same amino acid family or subgroup) to replace one or more amino acids of the reference polypeptide (for example, a peptide whose sequence has been published in the literature or sequence database or whose sequence has been determined via nucleic acid sequencing).

The “natural” or “wild-type” refers to a form found in nature. For example, a natural or wild-type peptide or polynucleotide sequence is a sequence present in living organisms, such as a DNA polymerase sequence that has not been intentionally modified by human.

The term “percent identity” or “homology” with respect to the nucleic acid or peptide sequences is defined as the percentage of nucleotide or amino acid residues in a candidate sequence that are identical to a known polypeptide after the sequences are aligned for the purpose of maximum percentage identity and vacancies are introduced when needed to achieve the maximum percentage homology. An N-terminal or C-terminal insertion or deletion should not be interpreted as affecting homology. Homology or identity at the nucleotide or amino acid sequence level can be determined via BLAST (Basic Local Alignment Search Tool) analyses by executing algorithms via programs of blastp, blastn, blastx, tblastn and tblastx (Altschul (1997), Nucleic Acids Res. 25, 3389-3402, and Karlin (1990), Proc. Natl. Acad. Sci. USA 87, 2264-2268), which is tailored for sequence similarity search. A method used for the BLAST program is that similar segments with and without gaps in a query sequence and a database sequence are first considered, then statistical significance of all identified matches is evaluated, and finally only those matches that meet a preselected significance threshold are summarized. For a discussion of basic issues in similarity searches in sequence databases, see Altschul (1994), Nature Genetics 6: 119-129. Search parameters for histograms, description, comparison, expectation values (namely, statistical significance threshold for reporting matches for database sequences), cutoff values, matrices, and filtering (low complexity) can be set by default. A default scoring matrix used for the blastp, blastx, tblastn and tblastx is BLOSUM62 matrix (Henikoff (1992), Proc. Natl. Acad. Sci. USA 89, 10915-10919), which is recommended for a query sequence with a length exceeding 85 units (nucleotide bases or amino acids).

This application is intended to cover functional equivalents or functional variants of the DNA polymerase in this application. The terms “functional equivalent” and “functional variant” are used interchangeably herein. The “functional equivalent” and “functional variant” can be obtained via, for example, substitution, insertion, or deletion (for example, conservative substitution) of one or more amino acids of the DNA polymerase in this application.

### Nucleic Acid, Nucleic Acid Construct, and Host Cell

This application also provides isolated nucleic acids, including sequences encoding the DNA polymerases in this application. This application also relates to an isolated polynucleotide encoding at least one functional domain of the DNA polymerase in this application. Generally, such functional domain includes one or more substitutions described herein.

The nucleic acid molecule in this application can be produced by using standard molecular biology techniques well known to persons skilled in the art in conjunction with sequence information provided herein. For example, the desired nucleic acid can be prepared via PCR or synthesized de novo by using a standard synthesis technique.

When used herein, the terms “nucleic acid”, “polynucleotide”, and “nucleic acid molecule” are used interchangeably and are intended to include DNA and RNA (for example, mRNA) as well as analogues of DNA or RNA produced by using nucleotide analogues. The nucleic acid molecule may be single-stranded or double-stranded, but is preferably double-stranded DNA. The “isolated nucleic acid” and “isolated polynucleotide” are used interchangeably herein and refer to DNA or RNA that is not directly adjacent to two coding sequences (one sequence at the 5′ end and the other sequence at the 3′ end) directly adjacent to the DNA or RNA in the natural genome of the organism from which the DNA or RNA originated. Therefore, the term encompasses, for example, recombinant DNA integrated into the vector, recombinant DNA integrated into the autonomously replicating plasmid or virus, recombinant DNA integrated into the genomic DNA of prokaryotes or eukaryotes, or recombinant DNA that exists as a separate molecule independent of another sequence (for example, a cDNA or genomic DNA fragment produced via PCR or restriction endonuclease treatment). The term also includes recombinant DNA that is part of a heterozygous gene, and the heterozygous gene encodes additional polypeptides.

This application also relates to a nucleic acid construct containing the nucleic acid, the nucleic acid may be operationally linked to a control sequence that allows the nucleic acid to replicate or express in the host cell, and the control sequence includes but is not limited to, a promoter, an enhancer, a terminator, or an origin of replication. The term “nucleic acid construct” herein refers to a segment that has been modified to contain nucleic acids that are combined and juxtaposed in a way that would not exist in nature. The nucleic acid construct can refer to an expression cassette, an expression vector, or a replication vector.

The expression vector may be any vector (for example, a plasmid or virus) that facilitates the recombinant DNA procedure and can elicit the expression of the nucleic acid sequence encoding the DNA polymerase in this application. The selection of vector usually depends on the compatibility of the vector with the host cell into which the vector is to be introduced. The vector can be a linear plasmid or circular plasmid. The vector can be an autonomously replicating vector. That is, the vector exists as an extrachromosomal entity whose replication is independent of chromosomal replication, for example, the plasmid, extrachromosomal element, mini-chromosome, or artificial chromosome. If fungal-derived host cells are used, a suitable additional nucleic acid construct can be, for example, 2 μ or pKD1 plasmid derived from yeast. Alternatively, an expression vector may be a vector that is integrated into the genome when introduced into the host cell and that replicates along with the chromosome into which the vector has been integrated.

This application also relates to a host cell, including the nucleic acid or nucleic acid construct in this application. The nucleic acid construct and the vector in this application can be designed to express the DNA polymerase in this application in a prokaryotic host cell or an eukaryotic host cell. Suitable host cells for expressing the polymerase in this application are well known in the art, including, but not limited to, bacterial cells such as Escherichia coli, Lactobacillus kefir (Lactobacillus kefir), Lactobacillus brevis (Lactobacillus brevis), Lactobacillus minor (Lactobacillus minor), streptomycete and Salmonella typhimurium (Salmonella typhimurium) cells; fungal cells such as yeast cells (such as Saccharomyces cerevisiae or Pichia pastoris (Pichia pastoris)); insect cells such as drosophila S2 and lepidoptera Sf9 cells; animal cells such as CHO, COS, BHK, 293 and Bowes melanoma cells; and plant cells. Suitable culture media and growth conditions for the foregoing host cells are well known in the art.

The polynucleotide for expressing the polymerase polypeptide can be introduced into the cell in various methods known in the art, including, but not limited to, electroporation, biological particle bombardment, liposome-mediated transfection, calcium chloride transfection, and protoplast fusion. Persons skilled in the art are aware of the various methods of introducing polynucleotides into cells.

### Kit

This application also relates to a kit, including a DNA polymerase, a nucleic acid, a nucleic acid construct, or a host cell in this application. The kit may include various reagents and containers for polynucleotide syntheses (including syntheses in PCR). The kit in this application may also include one or more of the following substances: a polynucleotide precursor, a primer, a buffer, an instruction for use, and a reference substance.

### Composition

This application also relates to a composition, including the DNA polymerase in this application. The composition may be, for example, a PCR reaction system, including, for example, a primer, a buffer, dNTP, a template and/or Mg²⁺.

### Method of Preparing DNA Polymerase

This application also relates to a method of preparing DNA polymerase.

In some embodiments, the method includes:
providing a chimera polypeptide including a first domain, encoded by a nucleotide sequence selected from nucleotide sequences denoted as SEQ ID NOs: 576 to 583, a second domain, encoded by a nucleotide sequence selected from nucleotide sequences denoted as SEQ ID NOs: 584 to 591, and a third domain, encoded by a nucleotide sequence selected from nucleotide sequences denoted as SEQ ID NOs: 592 to 599; and
optionally, introducing one or more amino acid substitutions selected from the following:
V5T/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V107I, F110Y, L126I, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, I198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V2521, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, I434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, I478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K,
where the positions are defined with reference to SEQ ID NO: 575; and
obtaining the DNA polymerase with DNA replication activity.

The chimera polypeptide including the first domain, the second domain and the third domain can be provided via seamless cloning.

In some embodiments, the method includes introducing, into the polypeptide denoted as SEQ ID NO: 575, one or more amino acid substitutions selected from the following:
V5T/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V1071, F110Y, L1261, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, I198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V2521, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, I434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, I478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K,
where the positions are defined with reference to SEQ ID NO: 575.

### Use of Nucleic Acid Amplification

This application also relates to a nucleic acid amplification method, including amplifying a DNA sequence by using the DNA polymerase in this application, the kit, the composition (the PCR reaction system) or the DNA polymerase prepared in the preparation method in this application.

In some embodiments, in the method, the nucleic acid is mixed with the DNA polymerase in this application or a biologically active fragment thereof under conditions suitable for amplifying the nucleic acid; and the nucleic acid is amplified via the polymerase chain reaction, an isothermal amplification reaction, recombinase polymerase amplification reaction, rolling circle amplification, or strand displacement amplification. The amplification includes amplification of the nucleic acid in a solution or on a solid support such as nucleic acid beads, flow cells, an nucleic acid array, or pores existing on a surface of the solid support. The polymerase chain reactions (PCR) include, but are not limited to, hot start PCR, landing PCR, nested PCR, inverse PCR, site-directed PCR mutagenesis, RT-PCR, RACE, multiplex PCR, asymmetric PCR, in situ PCR, quantitative PCR, whole-genome amplification, and error-prone PCR.

### Method for Improving a Property of a DNA Polymerase

This application also relates to a method for improving a property of a DNA polymerase.

In some embodiments, the method includes replacing a corresponding domain of a to-be-improved DNA polymerase with one or more domains encoded by one nucleotide sequence selected from nucleotide sequences denoted as SEQ ID NOs: 576 to 599.

In some embodiments, the method includes:
introducing, into a to-be-improved DNA polymerase, one or more amino acid substitutions selected from the following:
VST/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V107I, F110Y, L1261, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, I198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V2521, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, I434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, I478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K, where the positions are defined with reference to SEQ ID NO: 575.

The improved properties may be selected from one or more of a better extension characteristic, a better DNA binding characteristic, a better corrective activity, a better fidelity, a higher amplification speed, a better tolerance to an inhibitor, and a higher long fragment amplification capability. In some embodiments, the improved property is selected from one or more of the following: better Mg²⁺ tolerance, better SDS tolerance, better TE tolerance, and higher long fragment amplification capability.

### BRIEF DESCRIPTION OF DRAWINGS

The following drawings are for an illustration purpose only other than a limitation purpose.

FIG. 1 exemplarily shows an alignment diagram of polymerase sequences. A sequence alignment result is obtained after amino acid sequence alignment of a polymerase derived from thermophilic bacteria. Similarity is over 85%. Amino acid sequences marked with “*” are the same amino acid sequences, and amino acid sequences marked with “.” are different amino acid sequences. Amino acid sequences of 8 template polymerases are numbered 1 to 8.

FIG. 2 exemplarily shows a library establishment process of a chimera polymerase library. A block A can be derived from domains from 8 different sources, a block B is also derived from domains in templates 1 to 8, and a block C is also derived from domains in the templates 1 to 8. The nucleotide sequence is blocks A1 to A8, blocks B1 to B8, and blocks C1 to C8.

FIG. 3 exemplarily shows a Mg²⁺ tolerance test. Lane 1: 0 mM Mg²⁺; Lane 2: 2 mM Mg²⁺; Lane 3: 4 mM Mg²⁺; Lane 4: 6 mM Mg²⁺; Lane 5: 8 mM Mg²⁺; and Lane 6: 10 mM Mg²⁺. All concentrations are final concentrations of reactions. A source of Mg²⁺ can be either MgCl₂ or MgSO₄. The results show that the chimera polymerase can tolerate 0 mM Mg²⁺ to 10 mM Mg²⁺.

FIG. 4 exemplarily shows an SDS tolerance test. Lane 1: 0% SDS; Lane 2:0.00125% SDS; Lane 3:0.0025% SDS; Lane 4:0.005% SDS; Lane 5:0.01% SDS; Lane 6:0.02% SDS; Lane 7:0.04% SDS; and Lane 8:0.08% SDS. All concentrations are final concentrations of reactions. The results show that the chimera polymerase can tolerate 0.02% SDS.

FIG. 5 exemplarily shows a TE tolerance test. Lane 1: 0×; Lane 2:0.03125× TE; Lane 3: 0.0625× TE; Lane 4: 0.125× TE; Lane 5: 0.25× TE; Lane 6: 0.5× TE; Lane 7: 1' TE; and Lane 8: 2× TE. All concentrations are final concentrations of reactions. The results show that the chimera polymerase can tolerate 1× TE.

FIG. 6 exemplarily shows amplification of human gDNAs to different sizes. Lane 1: 1 kb; Lane 2: 2 kb; Lane 3: 3 kb; Lane 4: 4 kb; Lane 5: 5 kb; Lane 6 :6 kb; Lane 7: 7 kb; Lane 8: 8 kb; Lane 9: 9 kb; and Lane 10: 10 kb. The results show that the chimera polymerase can amplify long fragments.

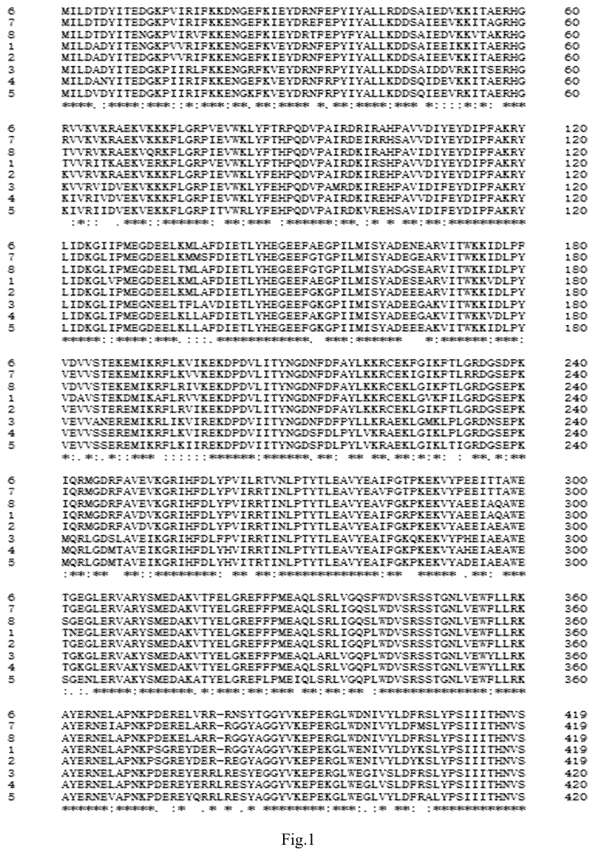

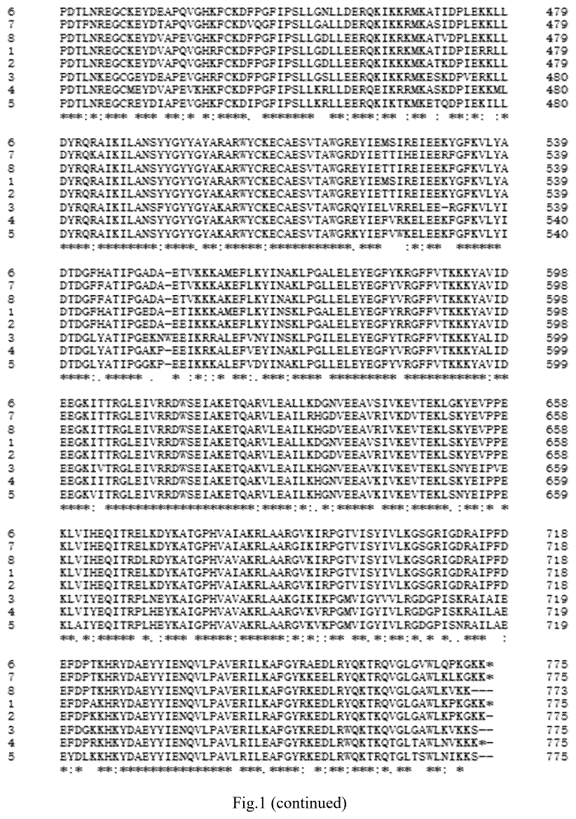

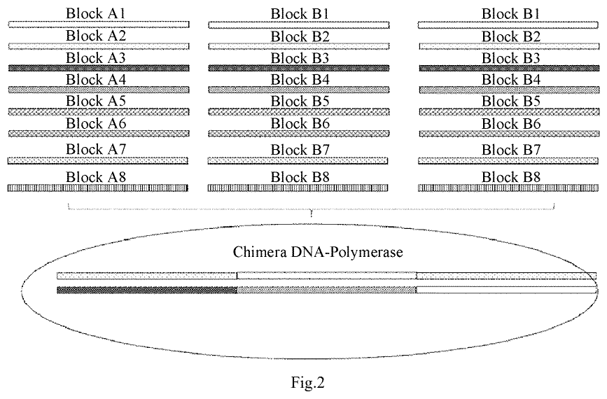

### DETAILED DESCRIPTION

### EXAMPLES

This application is further described with reference to specific examples. It should be understood that these examples are only used to illustrate the present invention and are not intended to limit the scope of the present invention. In the following example, a test method in which a detailed condition is not specified is usually performed under a regular condition or a condition recommended by a manufacturer. Unless otherwise stated, percentages and parts are calculated by weight.

Unless otherwise stated, materials and reagents used in the examples of the present invention are all commercially available products.

### Example 1: Preparation of a Chimera Polymerase

### 1. Clone Establishment

### 1.1. Primers

The primers (numbered as SEQ ID NOs: 600 to 647 in sequence) required for nucleotide sequences of the first domain with SEQ ID NOs: 576 to 583, the second domain with SEQ ID NOs: 584 to 591, and the third domain with SEQ ID NOs: 592 to 599 are shown in Table 1 below:

TABLE 1

| | |
|---|---|
| 576-F | CTTTAAGAAGGAGATATACATATGATTCTGGACGCTGACTATATT |
| 576-R | CATACGCTTTACGCAGCAGGAACCATTCAACCAGGTTACCG |
| 584-F | CTGCTGCGTAAAGCGTATGAACGCAACGAACTGGCACCGAATAAACCGTC |
| | CG |
| 584-R | GGGTTTCTTTGGCAATTTCACTCCAATCGCGACGCACAATTTCCAGACCG |
| 592-F | TGAAATTGCCAAAGAAACCCAGGCACGTGTTCTGGAAGCACTGCTGAAA |
| | G |
| 592-R | GTGGTGGTGCTCGAGTTACTTTTTACCCTTCGGTTTCAG |
| 577-F | AACTTTAAGAAGGAGATATACATAtgattctggatacggactata |
| 577-R | TCATACGCTTTACGCAGCAGaaaccattcgaccaggttacccg |
| 585-F | CTGCTGCGTAAAGCGTATGAACGCAACGAActggcgccgaataaaccggat |
| 585-R | TTTCTTTGGCAATTTCACTCCAATCacggcgcacgatttccagaccgc |
| 593-F | GTGAAATTGCCAAAGAAACCCAGGCAcgtgtcctggaagcaatcctg |
| 593-R | GTGGTGGTGGTGCTCGAGttactttttcactttcagccacgc |
| 578-F | TTTAAGAAGGAGATATACATATGATTCTGGACACGGACTATATTA |
| 578-R | GTTCATACGCTTTACGCAGCAGGAACCATTCAACCAGGTTACCCGT |
| 586-F | GCGTAAAGCGTATGAACGCAACGAAATTGCGCCGAATAAACCGGATGAA |
| 586-R | GTTTCTTTGGCAATTTCACTCCAATCACGGCGCACGATTTCCAGACC |
| 594-F | TGAAATTGCCAAAGAAACCCAGGCACGCGTTCTGGAAGCAATCCT |
| 594-R | GGTGGTGGTGGTGCTCGAGttaTTTCTTACCTTTCAGCTTCAG |
| 579-F | TTAAGAAGGAGATATACATATGATTCTGGATGCTGATTACATTA |
| 579-R | TTGCGTTCATACGCTTTACGCAGCAGATACCATTCCACCAGATTACC |
| 587-F | AGCGTATGAACGCAACGAACTGGCGCCGAATAAACCGGATGAAC |
| 587-R | TTTCTTTGGCAATTTCACTCCAATCGCGACGCACGATTTCCAGACCA |
| 595-F | GAGTGAAATTGCCAAAGAAACCCAGGCAAAGGTTCTGGAAGCAA |
| 595-R | GGTGGTGGTGGTGCTCGAGttaAGACTTTTTAACTTTCAGCC |
| 580-F | TTAAGAAGGAGATATACATAtgatcctggatgcggactacatt |
| 580-R | TCATACGCTTTACGCAGCAGgaaccattcgaccaggttaccgg |
| 588-F | TGCGTAAAGCGTATGAACGCAACGAActggcgccgaataaaccgtcgggc |
| 588-R | TTCTTTGGCAATTTCACTCCAATCgcgacgcacgatttccagacc |
| 596-F | AGTGAAATTGCCAAAGAAACCCAGGCAcgcgtcctggaagcaatcctgaaag |
| 596-R | GGTGGTGGTGGTGCTCGAGttattttttacctttcggtttcag |
| 581-F | CTTTAAGAAGGAGATATACATATGATCCTGGACACCGATTACATTAC |
| 581-R | GGTGGTGGTGGTGCTCGAGTTATTTCTTACCTTTCGGTTGCAG |
| 589-F | CTGCTGCGTAAAGCGTATGAAC |
| 589-R | TTCGTTGCGTTCATACGCTTTACG |
| 597-F | TGAAATTGCCAAAGAAACCCAGGCA |
| 597-R | GTTTCTTTGGCAATTTCACTCCAATC |
| 582-F | TTTAAGAAGGAGATATACATATGATCCTGGACGCAAACTACAT |
| 582-R | TCATACGCTTTACGCAGCAGATACCATTCCACCAGGTTACCGG |
| 590-F | GCGTAAAGCGTATGAACGCAACGAACTGGCACCGAATAAACCGGATG |
| 590-R | TTTCTTTGGCAATTTCACTCCAATCGCGACGAACGATTTCCAGACC |
| 598-F | GTGAAATTGCCAAAGAAACCCAGGCAAAAGTTCTGGAAGCAATCCT |
| 598-R | GTGGTGGTGGTGCTCGAGTTATTTCTTTTTCACATTCAGC |
| 583-F | TTTAAGAAGGAGATATACATATGATCCTGGACGTGGACTACA |
| 583-R | CATACGCTTTACGCAGCAGGAACCATTCAACCAGATTGCC |
| 591-F | TGCTGCGTAAAGCGTATGAACGCAACGAAGTGGCACCGAATAAACCGGAT |
| | GAA |
| 591-R | TTCTTTGGCAATTTCACTCCAATCGCGACGAACAATTTCCAGGC |
| 599-F | GGAGTGAAATTGCCAAAGAAACCCAGGCACGTGTTCTGGAAGCAATTCT |
| | GAAAC |
| 599-R | TGGTGGTGGTGGTGCTCGAGttaCGATTTTTTAATATTCAGCCATG |

1.2. Main Reagents: Plasmids were all plasmid templates stored in the laboratory, dNTP (RK20120, ABclonal), Pfu-fast 2× PCR Master Mix (RK20652, ABclonal), and 2× MultiF Seamless Assembly Mix (RK21020, ABclonal).

1.3. Main Instruments: PCR machine (Eastwin), ETC811; gel imager, Tanon 1600; electrophoresis apparatus, EPS 300; oscillator, VORTEX-5, Kylin-Bell; and NanoDrop 1000, Thermo.

### 2. Test Procedure

### 2.1. PCR Amplification

A reaction system was prepared, and then the system was quickly transferred to a PCR machine (Eastwin, ETC811) preheated 95° C. A 50 μL reaction system is shown in Table 2 below.

TABLE 2

| Components | Content | |
|---|---|---|
| ddH₂O | Diluted to 50 μL | |
| Upstream primer (10 μM) | 1 | μL |
| Downstream primer (10 μM) | 1 | μL |
| Template DNA | 10 | ng |
| Pfu-fast 2X PCR Master Mix | 25 | μL |

PCR reaction procedure

| | | |
|---|---|---|
| Temperature | Time | Quantity of cycles |

| | | | |
|---|---|---|---|
| 95° C. | 2 | min | 1 |
| 95° C. | 20 | s | |
| Tm-5° C. | 20 | s | 30 |
| 72° C. | 1 | min | |
| 72° C. | 5 | min | 1 |

| | |
|---|---|
| 4° C.-10° C. | ∞ |

### 2.2. Product Determination

After 10 μL of product was taken and 2 μL of 6× loading buffer was added and mixed well, 1% agarose gel electrophoresis was performed for determination at 150V for 45 minutes, and the gel imager was used to see if there were correct bands.

### 2.3. Product Purification

Purification was performed with a common DNA product purification kit (common DNA product purification kit, DP204, Tiangen Biotech (Beijing) Co., Ltd.).

2.3.1. Before use, absolute ethanol needed to be added to a rinse solution PW.

2.3.2. Column Equilibration Step: 500 μL of equilibration solution BL was added to an adsorption column CB2 (the adsorption column was put in the collection tube), the collection tube was centrifuged at 12000 rpm (˜13400×g) for 1 minute, a waste liquid in the collection tube was discarded, and the adsorption column CB2 was put back into the collection tube.

2.3.3. The volume of the PCR reaction solution or enzyme digestion reaction solution was

estimated, the binding solution PB with the volume 5 times the volume of the solution was add to the solution, and mixed well (paraffin oil or mineral oil DOES did not need to be removed). Note: If the volume of the PCR reaction solution was 50 μL (excluding the volume of paraffin oil), 250 μL of binding solution PB was added.

2.3.4. The solution obtained through the previous step was added to an adsorption column CB2 (the adsorption column was put in the collection tube), the collection tube was left standing at room temperature for 2 minutes, and centrifuged at 12000 rpm (˜13400×g) for 30 s to 60 s, a waste liquid in the collection tube was discarded, and the adsorption column CB2 was put into the collection tube. Note: The volume of adsorption column was 800 μL, and if the volume of sample was greater than 800 μL, the sample could be added in batches.

2.3.5. 600 μL of rinse solution PW was added to the adsorption column CB2 (It should be checked whether absolute ethanol was added before use), the adsorption column was centrifuged at 12000 rpm (˜13400×g) for 30 s to 60 s, the waste liquid in the collection tube was discarded, and the adsorption column CB2 was put into the collection tube. Note: If the purified DNA is used for salt-sensitive experiments, such as blunt-end ligation experiments or direct sequencing, it is recommended that PW be added and left standing for 2 minutes to 5 minutes before centrifugation.

2.3.6. The adsorption column CB2 was put back into the collection tube, the collection tube was centrifuged at 12000 rpm (˜13400×g) for 2 minutes, and the rinse solution was removed to the maximum extent. The adsorption column CB2 was left standing at room temperature for a few minutes and air-dried thoroughly to prevent residual rinse solution from affecting the subsequent test. Note: Ethanol residues in the rinse solution can affect subsequent enzymatic reaction (enzyme digestion, PCR, and the like) tests

2.3.77. The adsorption column CB2 was put into a clean centrifuge tube, 30 μL of elution buffer EB was added dropwise to the middle of an adsorption membrane and was left standing for 2 minutes at room temperature. The centrifuge tube was centrifuged at 12000 rpm (˜13400×g) for 2 minutes to collect the DNA solution.

### 2.4. Quantitative Determination

The purified PCR product was taken and 1 μL of the purified PCR product was added to the Nanodrop 1000 for concentration determination.

### 2.5. Ligation

The ABclonal MultiF Seamless Assembly Mix (RK21020) was used for ligation. The specific reaction system is shown in Table 3 below:

TABLE 3

| Components | Dosage | |
|---|---|---|
| Total amount of inserted DNA | 1 | pmol |
| Amount of carrier | 0.3 | pmol |
| 2X MultiF Seamless | 10 | μL |
| ddH₂O | Diluted to 20 μL | |
| Total volume | 20 | μL |

Reaction process

| Fragment assembly | 24 fragments | |
|---|---|---|
| Reaction temperature | 50° | C. |
| Reaction time | 60 | min |

### 2.6. Transformation

2.6.1. Competent cells used for cloning (C2566, ABclonal) thawed on ice.

2.6.2. 10 μL of the assembly product was added to 100 μL of competent cells, a tube wall

was flicked for even mixing (oscillation should be avoided), a resulting mixture was left standing on ice for 30 minutes; and a volume of the assembly product after transformation should not exceed ⅙ of a volume of the used competent cells.

2.6.3. After being subjected to heat shock in water bath at 42° C. for 45 seconds, the resulting mixture was immediately put on ice for cooling for 2 to 3 minutes.

2.6.4. 900 μL of SOC or LB culture medium (containing no antibiotics) was added and the

resulting mixture was oscillated for even mixing at 37° C. for 1 hour (rotational speed: 200 rpm to 250 rpm).

2.6.5. A corresponding resistant LB plate was preheated in an incubator at 37° C.

2.6.6. The resulting mixture was centrifuged at 5000 rpm for 5 minutes, 900 μL of the supernatant was discarded, the bacteria was resuspended, and a resuspension was gently spread evenly on the plate containing the corresponding resistant substance with a sterile spreader stick.

2.6.7. The resulting mixture was put upside down and incubated in the incubator at 37° C. for 12 hours to 16 hours.

### 2.7. Sequencing

After overnight culture, hundreds of monoclonals can be formed on the plate, and clones on the plate in the negative control group after transformation should be significantly outnumbered by the former; and a few monoclonals were selected for determination in a first-generation sequencing method. If a sequencing result of the plasmid was correct, the plasmid was saved and expressed.

### 2. High-Throughput Expression and Purification

### 2.1. Main Reagents and Materials

(1) 96-well PCR plate (Axygen, catalog number: PCR-96m2-hs-c, no sterilization required), 48-well deep-well plate (Sangon Biotech, catalog number: F600480-0001, autoclave sterilized and dried before use), 96-well deep-well plate (NEST, catalog number: 503001, no sterilization required) and matching silicone lids (sterilization required), 96-well filter plate (Sangon Biotech, catalog number: B615006, no sterilization required), quartz sand, DEAE padding (GE), DNase I (Abclonal, catalog number: RK20538), and 100 μL PCR 8-tube strips (Axygen, catalog number: PCR-0108-LP-RT-C).
(2) LB liquid medium (ABclonal)
(3) IPTG, Amp, 50% glycerol (ABclonal)

### 2.2. Main Instruments

Super clean bench (Airtech, SW-CJ-1FD), refrigerated centrifuge (Cence, L530R), small-sized thermostatic oscillator (Crystal Technology & Industries, Inc., IS-RSD81), PCR machine (Eastwin, ETC811), high-throughput tissue grinder (Wonbio, Wonbio-96), microplate reader (SYNERG, H1), precision constant temperature sink (Shanghai Yiheng Instruments Co., Ltd., BWS-12), and so on.

### 2.3. Test Steps

### 2.3.1. Recombinant Plasmid Transformation

(1) The recombinant plasmids were sorted, 47 vials were put in each box and arranged in order.
(2) A 96-well PCR plate (unopened), 200 μL yellow pipette tips, and an alcohol sprayer were put onto an aseptic operation table in advance, and were sterilized via ultraviolet radiation for 30 minutes.
(3) 2566 competent cells were removed from −80° C. and put on ice to thaw (the quantity was calculated based on the number of transformations).
(4) A metal block of a 96-well plate was taken and put on ice, the 96-well PCR plate was put in the metal block, the competent cells were subpackaged, 50 μL of the competent cells was added to each well and the pipette tips should be prevented from contamination.
(5) The 96-well PCR plate was marked, and 0.5 L to 1 μL of the plasmids with corresponding numbers were separately added and the PCR plate was put on ice for 20 minutes to 30 minutes.
(6) A lid of the PCR machine was opened and the PCR plate was incubated at 42° C. for 90 seconds.
(7) The PCR plate was put on ice for 3 minutes.
(8) 100 μL of LB (at a concentration of 100 μg/mL) with Amp resistance was added, and

the PCR plate was incubated at 37° C. for 30 minutes to 45 minutes, and then transferred to a 96-well deep-well plate (600 μL to 1 mL of LB containing Amp was added), and the resulting mixture was oscillated at 700 rpm and cultured overnight at 37° C.
(9) A record form was made for strain storage. The table content included: strain name, corresponding well positions of each strain, 96-well plate number, culture time, operator, and various abnormal situations (no growth, wrong sample added, or the like).

### 2.3.2. Inducible Expression of High-Throughput Proteins

2 48-well deep-well plates were taken and 4 mL of LB medium containing Amp (at a concentration of 100 μg/mL) was added to each well.

100 μL of the bacteria solution cultured overnight was taken and transferred to a 48-well deep-well plate, the deep-well plate was marked, put on a thermostatic oscillator at 37° C., and oscillated at 700 rpm, an equal volume of 50% glycerol (sterilization) was added to the bacteria residue in the 96-well plate, and the bacteria were cryopreserved and stored at −20° C.

After 2.5 hours to 3 hours of incubation, 300 μL of bacteria solution were randomly taken from 4 wells on the outermost side of the 48-well plate, OD₆₀₀ was measured by using the microplate reader with ultrapure water as a control group. When OD₆₀₀ reached 0.8 to 0.9, IPTG at a final concentration of 0.5 mM (2 μL of 1 M/L IPTG solution was added) was added to each well, and the reaction was induced at room temperature overnight.

The next day, after overnight induction, the 48-well plate was put into a horizontal centrifuge and centrifuged at 4000 rpm for 30 minutes, the supernatant was immediately removed and discarded, and the plate was spun dry with great force (bacteria were attached very firmly to the bottom of the 48-well plate and did not fall off). The plate was stored −20° C.

A record form was made for inducible expression. The table content included: strain name, corresponding well positions of each strain, plate number, culture time, operator, and abnormal situations (no growth, no induction, wrong positions, or the like).

### 2.3.3. High-Throughput Crushing

(1) The 48-well plate containing bacteria was removed from the freezer and thawed, the water bath cauldron was turned on in advance and the temperature was set to 80° C., 700 μL of lysis buffer (20 mM Tris-HCl, 2.5 mM MgCl₂, pH=7.5@25° C.) was added to each well, and the bacteria was thoroughly suspended by repeatedly pipetting by using a multichannel pipette.
(2) The suspended bacteria solution was transferred to a 96-well deep-well plate (NEST).
(3) The 96-well plate was put at −80° C. for 45 minutes until completely frozen. The plate was removed, put on a small-sized oscillator at 37° C., and oscillated at 700 rpm for 40 minutes until completely thawed, and then the 96-well plate was put in an 80° C. water bath for 10 minutes.
(4) Step (3) was performed again.
(5) The 96-well plate was removed from the water bath cauldron, immediately put into −80° C. for 45 minutes until completely frozen, then removed and put on the small-sized thermostatic oscillator at 37° C., and oscillated at 700 rpm for 40 minutes until completely thawed. Freeze-thaw time in the foregoing process needs to be adjusted according to an actual situation.
(6) 100 μL of quartz sand was added to each well (via 100 μL PCR 8-tube strips), covered with a silica gel cap (sterilized), and put in the high-throughput tissue grinder (Wonbio), a metal cap was tightened, the resulting mixture was oscillated at 60 Hz for 60 s for a total of 5 times, the cover was opened to dissipate heat for at least 5 minutes after each oscillation to prevent the lysate solution from overflowing due to overheating after continuous oscillation.
(7) The resulting mixture was centrifuged at 4000 rpm for 30 minutes, a supernatant was transferred to a 96-well filter plate with the multichannel pipette (with a volume adjusted 500 μL), a liquid was gathered with a 96-well deep-well plate below, and the two plates were pasted and fixed with an adhesive tape, and centrifuged at 3000 rpm for 2 minutes to remove impurities (the precipitate was pipetted during the transfer).

### 2.3.4. High-Throughput Purification

(1) After 1.5 μL of DNase I (Abclonal) was added to each well, the 96-well plate was put on a small-sized oscillator at room temperature and oscillated at 700 rpm for 5 minutes for even mixing, and then the plate was kept in a 37° C. water bath for 2 hours.
(2) The 96-well plate was removed, DTT at a final concentration of 2 mM was added to each well (1 μL of DTT was added to 1 M/L stock solution), water bath temperature was set to 80° C., and after the temperature was stable, the 96-well plate was put in the water bath and inactivated for 30 minutes. After the plate was removed, the plate was cooled completely at 4° C. and centrifuged at 4000 rpm for 2 minutes.
(3) Preparation of DEAE Sepharose:

Equilibration buffer: 10 mM Tris-HC1, 190 mM Kcl, and 0.1 mM EDTA.

A suspension with a volume of 150 μL was added to the 96-well filter plate (pure padding volume=suspension volume×padding ratio), the liquid was gathered with a 96-well deep-well plate below, the resulting mixture was centrifuged at 3000 rpm for 2 minutes, the padding storage solution was discarded, 600 μL of ultrapure water was added to each well of the 96-well filter plate, the resulting mixture was centrifuged at 3000 rpm for 2 minutes, washed with water once, washed with the equilibration buffer once, and transferred to a new 96-well deep-well plate for loading.
(4) DEAE loading: A KCl solution (at final concentration of 190 mM, 34 μL of 3M KCl solution (Vetec) was added to each well) was added to an inactivated protein sample, put in a small-sized oscillator at room temperature, and oscillated at 700 rpm for 5 minutes to be mixed well. The samples were transferred to the balanced DEAE padding (in the 96-well filter plate) with the multichannel pipette in a one-to-one correspondence with the positions of the wells, then the 96-well filter plate and the 96-well deep-well plate for gathering liquid below were fixed with an adhesive tape, and put into a small-sized thermostatic oscillator at room temperature and oscillated at 1000 rpm for 2 hours. The samples were centrifuged at 3000 rpm for 2 minutes to obtain purified protein samples, and the purified protein samples were temporarily stored at 4° C.

### 2.3.5. SDS-PAGE Determination

Samples were taken from 96-well PCR plate for testing.
1. Sample Preparation: 7 μL of proteins+7 μL of 2×SDS loading buffer
2. Electrophoresis Conditions: Each of 15 wells was loaded with 10 μL of SDS-PAGE gel (a concentration of separation gel was 12%).

A constant voltage was set to 200V, electrophoresis was conducted for 36 minutes, and the samples were stained with Coomassie brilliant blue for 2 minutes, decolorized and photographed.
3. Result Processing: The sample numbers and the positions on the plate (including numbers of the 96-well plates) were marked on the electrophoretogram, and expression states were collected.

### 2.3.6. Protein Concentration Determination

1. The Bradford working solution was removed from the 4° C. freezer and put at room temperature before use.
2. A 12-tube strip was taken and a standard substance was diluted according to Table 4 below.

TABLE 4

| | | | | | | | | | | | |
|---|---|---|---|---|---|---|---|---|---|---|---|
| 1 X PBS (μL) | 100 | 95 | 90 | 80 | 70 | 60 | 50 | 40 | 25 | 10 | 0 |
| 2 mg/mL BSA (μL) | 0 | 5 | 10 | 20 | 30 | 40 | 50 | 60 | 75 | 90 | 100 |
| Final concentration of BSA (mg/mL) | 0 | 0.1 | 0.2 | 0.4 | 0.6 | 0.8 | 1 | 1.2 | 1.5 | 1.8 | 2 |

3. A 96-well microplate was taken, 10 μL of standard substance and sample were added to each well, and 1 duplication was configured for each standard substance and sample.
4. 200 μL of the Bradford working solution was added to each well.
5. The microplate was put into a microplate reader and oscillated for 30 s, and was left standing for 5 minutes to determine A595.
6. An average of A595 in the standard group was used as the abscissa, and the corresponding protein concentration was used as the ordinate, and a standard curve was plotted in excel software.
7. The protein concentration of the sample was calculated based on the average of A595 and the excel curve.

### Example 2 Application Test of Chimera Polymerase

The sequence tested in this example had SEQ ID NO: 278, and the magnesium ion tolerance, TE buffer tolerance, SDS tolerance, long fragment amplification capability and the polymerase were specifically detected.

### 1. Templates and Primers

The templates used in this example were Escherichia coli gDNA and human gDNA, and the target gene of Escherichia coli gDNA was 16S, and had a size of 400 bp to 500 bp (templates extracted from Tiangen kit); and a size of a target fragment of human gDNA was 0.5 kb to 10 kb (templates extracted from Tiangen kit).

The primers (SEQ ID NOs: 648 to 669, synthesized by Sangon Biotech) used in the test are shown in Table 5 below:

TABLE 5
Amplification primers

| | | | Product | |
|---|---|---|---|---|
| Template | Primer | Sequence | size | |
| E. coli | F | GACGCTCTTCCGATCTTATGGTAATTGTGTGCCA | 500 | bp |
| | | GCMGCCGCGGTAA | | |
| | R | TGTGCTCTTCCGATCTAGTCAGTCAGCCGGACT | | |
| | | ACHVGGGTWTCTAAT | | |
| Human | F | CCTCATTTGGGGAGGGGTTATCT | 1 | kb |
| | R | GGGGCACCTTCTCCAACTCATACT | | |
| Human | F | GGGGCACCTTCTCCAACTCATACT | 2 | kb |
| | R | CCTGAAACAAGGTTGTGGCATAGC | | |
| Human | F | ATGAGGCAAGATATGAGTGC | 3 | kb |
| | R | AATCTCTGGCCAGATGTGTTC | | |
| Human | F | ATGAGGCAAGATATGAGTGC | 4 | kb |
| | R | GTTCTAGAGTACAATGTGCACAATG | | |
| Human | F | ATGAGGCAAGATATGAGTGC | 5 | kb |
| | R | TGACCAAACTCCATCTTCTCTC | | |
| Human | F | ATGAGGCAAGATATGAGTGC | 6 | kb |
| | R | ATGACACTGATCCAGGGAC | | |
| Human | F | ATGAGGCAAGATATGAGTGC | 7 | kb |
| | R | TTGCCATGTAACATACTTGTACAC | | |
| Human | F | ATGAGGCAAGATATGAGTGC | 8 | kb |
| | R | TGGCTCATGTATCACACAATG | | |
| Human | F | ATGAGGCAAGATATGAGTGC | 9 | kb |
| | R | ACCTAAACCTTGCAATTGGG | | |
| Human | F | ATGAGGCAAGATATGAGTGC | 10 | kb |
| | R | TTGGCTATTCTTGTTGCTG | | |

### 2. PCR Reaction System

The foregoing templates and primers, dNTPs (source), enzyme, buffer (source), and PCR tubes were put on ice to prepare the reaction system shown in Table 6 below.

TABLE 6
PCR Reaction System

| Components | 50 μL | |
|---|---|---|
| 5X reaction buffer | 25 | μL |
| Upstream primer (10 μM) | 1 | μL |
| Downstream primer (10 μM) | 1 | μL |
| dNTP (10 mM) | 1 | μL |
| DNA template | 10 | ng |
| Polymerase | 50 | ng |

| | |
|---|---|
| Nuclease-free water | Diluted to 50 μL |

Components of 1× buffer solution: 20 mM Tris-HCl, pH of 8.8, 10 mM (NH₄)₂SO₄, 1.5 mM MgSO₄, and 100 mM KCl. A final concentration of dNTPs was 200 uM; and a final concentration of primers was 200 nM. The dosage of the template was 10 ng.

### 3. Reaction Process

The PCR reaction process is shown in Table 7 below. The following process was executed in a PCR machine (Eastwin, E811):

TABLE 7
PCR Reaction Process

| Steps | Temperature | Time | | Quantity of cycles |
|---|---|---|---|---|
| Pre-denaturation | 98° C. | 45 | s | 1 |
| Denaturation | 98° C. | 10 | s | 30 |
| annealing | 60° C. | 30 | s | |
| Extension* | 72° C. | 5 | min | |
| Final extension | 72° C. | 1-5 | min | 1 |

| | | | |
|---|---|---|---|
| Holding | 4° C.-12° C. | ∞ | 1 |

### 4. Test Results

4.1. For a tolerance range for Mg2+, a final concentration was 0 mM MgCl₂ to 10 mM MgCl₂. The results are shown in FIG. 3.

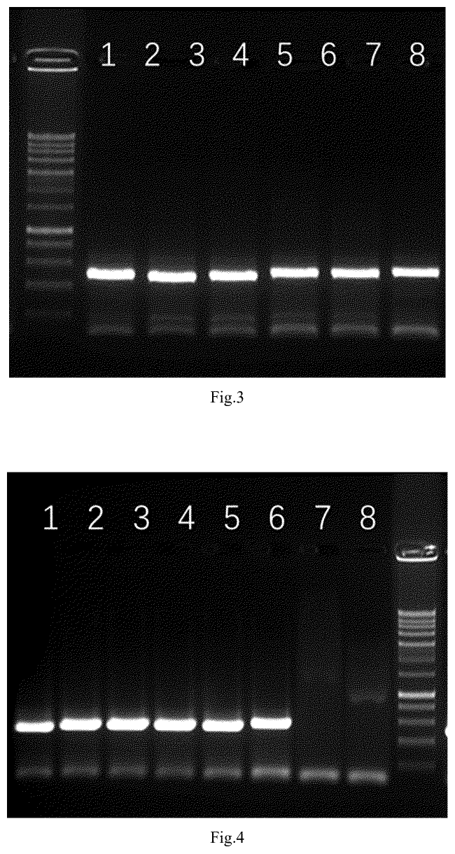

4.2. For a tolerance range for SDS, a final concentration was 0% SDS to 0.02% SDS. The results are shown in FIG. 4.

4.3. For a tolerance range for a TE buffer was 0 to 1×. The results are shown in FIG. 5.

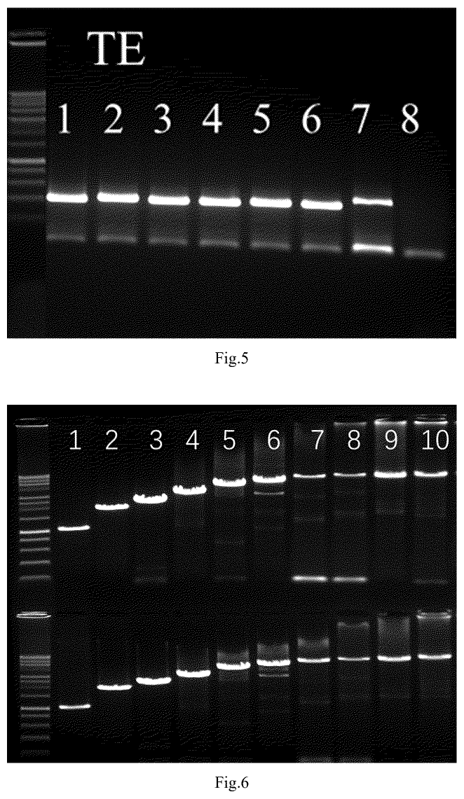

4.4. For amplification of human gDNA, a maximum of 10 kb human gDNA could be amplified. The results are shown in FIG. 6.

The specific method and composition described herein represent preferred embodiments and are exemplary and are not intended to limit the scope of the present invention. Persons skilled in the art can figure out other objectives, aspects and embodiments after considering this specification and conclude that the objectives, aspects and embodiments are included within the spirit of the present invention defined by the scope of the claims. It is apparent to persons skilled in the art that various substitutions and modifications can be made to the present invention disclosed herein without departing from the scope and spirit of the present invention. The present invention illustratively described herein may be suitably practiced in the absence of any elements or limitations, which are not specifically disclosed herein as being necessary. Therefore, for example, in each example herein, any one of the terms such as “comprise”, “include”, and “contain” within the embodiments or examples of the present invention shall be interpreted in a broad sense without limitation. The steps described herein may be practiced in different step sequences, which are not necessarily limited to the step sequences indicated herein or in the claims. Unless otherwise expressly indicated in the context, “a” and “the” include plural forms and the plural forms include singular forms. In no event shall any statement made by any examiner or any other officer or employee of the Patent and Trademark Office be construed as a limitation on the patent unless such statement is explicit and is expressly adopted by the applicant in responsive writing without conditions or reservation.

The present invention is described broadly and generally. Each of narrower category and subcategory groups falling within the scope of the general disclosure range also forms a part of the present invention. The adopted terms and expressions are used as descriptive terms other than limitative terms, and the use of such terms and expressions is not intended to exclude any equivalents of the features shown and described, or parts thereof. However, it can be understood that various modifications may be made within the claimed scope of the present invention. Therefore, it should be understood that, although the present invention is specifically disclosed through preferred embodiments and optional features, persons skilled in the art may make modifications and variations to the concepts disclosed herein, and such modifications and variations may be regarded as limitations on the present invention, and fall within the scope of the present invention that is defined in the appended claims.

Nucleotide Sequence
SEQ ID NO: 575
atgattttagatgtggattacataactgaagaaggaaaacctgttattag
gctattcaaaaaagagaacggaaaatttaagatagagcatgatagaactt
ttagaccatacatttacgctcttctcagggatgattcaaagattgaagaa
gttaagaaaataacgggggaaaggcatggaaagattgtgagaattgttga
tgtagagaaggttgagaaaaagtttctcggcaagcctattaccgtgtgga
aactttatttggaacatccccaagatgttcccactattagagaaaaagtt
agagaacatccagcagttgtggacatcttcgaatacgatattccatttgc
aaagagatacctcatcgacaaaggcctaataccaatggagggggaagaag
agctaaagattcttgccttcgatatagaaaccctctatcacgaaggagaa
gagtttggaaaaggcccaattataatgattagttatgcagatgaaaatga
agcaaaggtgattacttggaaaaacatagatcttccatacgttgaggttg
tatcaagcgagagagagatgataaagagatttctcaggattatcagggag
aaggatcctgacattatagttacttataatggagactcattcgacttccc
atatttagcgaaaagggcagaaaaacttgggattaaattaaccattggaa
gagatggaagcgagcccaagatgcagagaataggcgatatgacggctgta
gaagtcaagggaagaatacatttcgacttgtatcatgtaataacaaggac
aataaatctcccaacatacacactagaggctgtatatgaagcaatttttg
gaaagccaaaggagaaggtatacgccgacgagatagcaaaagcctgggaa
agtggagagaaccttgagagagttgccaaatactcgatggaagatgcaaa
ggcaacttatgaactcgggaaagaattccttccaatggaaattcagcttt
caagattagttggacaacctttatgggatgtttcaaggtcaagcacaggg
aaccttgtagagtggttcttacttaggaaagcctacgaaagaaacgaagt
agctccaaacaagccaagtgaagaggagtatcaaagaaggctcagggaga
gctacacaggtggattcgttaaagagccagaaaaggggttgtgggaaaac
atagtatacctagattttagagccctatatccctcgattataattaccca
caatgtttctcccgatactctaaatcttgagggatgcaagaactatgata
tcgctcctcaagtaggccacaagttctgcaaggacatccctggttttata
ccaagtctcttgggacatttgttagaggaaagacaaaagattaagacaaa
aatgaaggaaactcaagatcctatagaaaaaatactccttgactatagac
aaaaagcgataaaactcttagcaaattctttctacggatattatggctat
gcaaaagcaagatggtactgtaaggagtgtgctgagagcgttactgcctg
gggaagaaagtacatcgagttagtatggaaggagctcgaagaaaagtttg
gatttaaagtcctctacattgacactgatggtctctatgcaactatccca
ggaggagaaagtgaggaaataaagaaaaaggctctagaatttgtaaaata
cataaattcaaagctccctggactgctagagcttgaatatgaagggtttt
ataagaggggattcttcgttacgaagaagaggtatgcagtaatagatgaa
gaaggaaaagtcattactcgtggtttagagatagttaggagagattggag
tgaaattgcaaaagaaactcaagctagagttttggagacaatactaaaac
acggagatgttgaagaagctgtgagaatagtaaaagaagtaatacaaaag
cttgccaattatgaaattccaccagagaagctcgcaatatatgagcagat
aacaagaccattacatgagtataaggcgataggtcctcacgtagctgttg
caaagaaactagctgctaaaggagttaaaataaagccaggaatggtaatt
ggatacatagtacttagaggcgatggtccaattagcaatagggcaattct
agctgaggaatacgatcccaaaaagcacaagtatgacgcagaatattaca
ttgagaaccaggttcttccagcggtacttaggatattggagggatttgga
tacagaaaggaagacctcagataccaaaagacaagacaagtcggcctaac
ttcctggcttaacattaaaaaatcctag

1. A chimera DNA polymerase with DNA replication activity, comprising:
a first domain, encoded by a nucleotide sequence selected from nucleotide sequences denoted as SEQ ID NOs: 576 to 583 or a nucleotide sequence that shares a sequence identity of at least 70%, 75%, 80%, 85%, 90%, 95%, 97%, 98% or 99% with one of the nucleotide sequences denoted as SEQ ID NOs: 576 to 583;
a second domain, encoded by a nucleotide sequence selected from nucleotide sequences denoted as SEQ ID NOs: 584 to 591 or a nucleotide sequence that shares a sequence identity of at least 70%, 75%, 80%, 85%, 90%, 95%, 97%, 98% or 99% with one of the nucleotide sequences denoted as SEQ ID NOs: 584 to 591; and
a third domain, encoded by a nucleotide sequence selected from nucleotide sequences denoted as SEQ ID NOs: 592 to 599 or a nucleotide sequence that shares a sequence identity of at least 70%, 75%, 80%, 85%, 90%, 95%, 97%, 98% or 99% with one of the nucleotide sequences denoted as SEQ ID NOs: 592 to 599.

2. The chimera DNA polymerase according to claim 1, wherein when compared with reference polypeptide denoted as SEQ ID NO: 575, an amino acid sequence of the DNA polymerase comprises one or more amino acid substitutions corresponding to amino acids at the following positions:
5, 6, 11, 15, 16, 18, 22, 24, 25, 28, 30, 33, 35, 36, 38, 43, 47, 49, 50, 51, 52, 54, 56, 57, 61, 62, 64, 65, 66, 67, 68, 72, 73, 80, 81, 84, 88, 89, 90, 94, 96, 99, 100, 102, 104, 107, 110, 126, 127, 132, 136, 137, 138, 139, 140, 153, 154, 158, 165, 166, 167, 169, 176, 180, 182, 183, 185, 186, 188, 189, 193, 194, 195, 196, 197, 198, 199, 206, 210, 213, 216, 217, 220, 223, 226, 228, 230, 231, 232, 233, 236, 238, 241, 244, 247, 248, 251, 252, 261, 262, 265, 268, 282, 285, 286, 292, 293, 296, 297, 301, 302, 303, 304, 310, 318, 320, 324, 327, 331, 334, 337, 340, 341, 356, 367, 373, 374, 375, 377, 378, 379, 383, 384, 386, 395, 399, 400, 401, 403, 406, 407, 408, 409, 410, 424, 426, 430, 434, 437, 439, 441, 446, 447, 455, 456, 459, 463, 466, 467, 470, 471, 472, 475, 477, 478, 479, 485, 494, 499, 502, 508, 520, 524, 525, 526, 527, 529, 532, 533, 540, 545, 546, 552, 553, 554, 556, 557, 559, 560, 562, 565, 566, 570, 575, 585, 588, 597, 604, 605, 626, 631, 633, 634, 636, 642, 646, 652, 653, 656, 658, 662, 664, 670, 672, 673, 677, 683, 690, 692, 694, 695, 698, 701, 703, 706, 708, 710, 712, 713, 717, 718, 719, 721, 723, 724, 727, 743, 747, 752, 753, 755, 758, 762, 764, 767, 768, 771, 772, 774, and 775,
where the positions are defined with reference to SEQ ID NO: 575.

3. The chimera DNA polymerase according to claim 2, wherein the amino acid substitution is selected from one or more of the following:
VST/A, D6N, E11N/D, V15I, I16V, I18V/L, E22N, G24K/E, K25R/E, I28V, H30Y, T33Y/E/N, R35E, P36E/H/M, 138F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V107I, F110Y, L126I, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, I198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V252I, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, 1434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, I478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V692I, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K.

4.-5. (canceled)

6. The chimera DNA polymerase according to claim 1, wherein the DNA polymerase shares a sequence identity of at least 70%, 75%, 80%, 85%, 90%, 95%, 97%, 98% or 99% with SEQ ID NO: 575.

7. The chimera DNA polymerase according to claim 1, comprising an amino acid sequence, wherein the amino acid sequence shares a sequence identity of at least 70%, at least 75%, at least 80%, at least 85%, at least 90%, at least 95%, at least 97%, at least 98%, at least 99%, or 100% with any one of the amino acid sequences denoted as SEQ ID NOs: 1 to 574 .

8. (canceled)

9. A nucleic acid, comprising a sequence encoding the chimera DNA polymerase according to claim 1.

10. A nucleic acid construct, comprising the nucleic acid according to claim 9.

11. A host cell, comprising the nucleic acid according to claim 9.

12. A kit, comprising the chimera DNA polymerase according to claim 1.

13. A composition, comprising the chimera DNA polymerase according to claim 1.

14.-15. (canceled)

16. A method of amplifying nucleic acid, comprising amplifying a DNA sequence by using the chimera DNA polymerase of claim 1.

17. A method for improving a property of a DNA polymerase, wherein the method comprises:
replacing a corresponding domain of a to-be-improved DNA polymerase with one or more domains encoded by one nucleotide sequence selected from nucleotide sequences denoted as SEQ ID NOs: 576 to 599.

18. A method for improving a property of a DNA polymerase, wherein the method comprises:
introducing, into a to-be-improved DNA polymerase, one or more amino acid substitutions selected from the following:
VST/A, D6N, E11N/D, V15I, I16V, I18V/L, E22N, G24K/E, K25R/E, I28V, H30Y, T33Y/E/N, R35E, P36E/H/M, I38F, R43K, K47Q/K/A, E49D, E50S, I51V, K52R, I54V, G56S/A, E57K/G, K61T/R, I62V, R64K/T, I65V, V66T/I/K, D67K/R, V68A, E72Q/K, K73R, I80V, T81E, K84R, E88T, H89R, P90F, P94E/Q, I96M, K99E/R, V100I, E102S/R/A, P104S, V107I, F110Y, L126I, I127V, E132N/D, K136T, I137F/L/M, L138M, A139S, F140V, G153A, K154E/T, I158L, E165G, N166S/G/E, E167G, K169R, I176V, Y180F, E182D, V183A, S185A, S186N/T, R188K, E189D, R193A, F194L, L195I, R196K, I197V, 1198V, R199K, I206L, N210D, S213N/D, F216L, P217A, A220L/V/K, A223C, L226F/I, I228M/V, L230F, T231P/I, I232L, G233R, G236N, E238K, I241M, I244L/M, M247S/R, T248L/F, E251D, V252I, Y261F, H262P, T265L/R, I268V, I282V, K285T/R, P286Q, A292P, D293H/E, A296T, K297Q/T/E, S301T, G302N, E303K, N304G, K310R, A318V, Y320F, K324R, F327L, I331A, S334A, V337I, P340S, L341F, F356Y, V367L, S373D, E374G/K, E375K/R, Y377L, Q378V/A/E/D, R379E, E383G/N, S384G, T386A/E, KR395R, E399D, N400G, I401L, Y403S, F406Y, R407K/M/H, A408S/D/F/G/P/R/T, L409S/D/F/G/P/R/T/A, Y410S/D/F/G/P/R/T/A, L424F, L426K/R, K430G/M/R, 1434E/T/V, Q437E, G439K, K441R, I446V/F, P447Q, G455K, H456N/A/S/R/D, E459D, K463E, T466R/K, K467R, E470A, T471S, Q472I/V/K, I475L/V, K477R, 1478R/K, L479M, K485R, F494Y, G499A, K502R, K508R, K520D/Q/E, L524M/T/F, V525T/S, W526R/I, K527H/R, L529I, K532R, F533Y/R, I540A, L545V/F/I, Y546V/I/A/T, G552E/A, E553K/D, S554N/P/D, E556T, I557V, K559R, K560R, L562K/M, V565L, K566N/E/D, S570A, L575A, K585V/R/T, F588L, V597L, I604V/T, I605V/T, R626K, I631L, K633R, H634D, D636N, R642K/S, E646D, A652G/S, N653K, I656V, P658V, A662V, Y664H, P670E/D, H672N/K/R, E673D, I677T, V683I, K690R, V6921, I694V, R695K, M698T, G701S, I703V, R706K, D708S, P710R, S712G, N713K/D, L717A/P, A718I/F, E719D, Y721F, P723G/L, K724T/A/R, K727R, L743E, E747R/K, R752K, K753R/A, D755E, Y758W, R762K, V764T, G767T, S768V/A, N771Q/K, I772L/V/P, K774G, and S775K,
where the positions are defined with reference to SEQ ID NO: 575.

19. The method according to claim 178, wherein the improved property is selected from one or more of the following: better Mg²⁺ tolerance, better SDS tolerance, better TE tolerance, and higher long fragment amplification capability.

20. A host cell, comprising the nucleic acid construct according to claim 10.

21. A method of amplifying nucleic acid, comprising amplifying a DNA sequence by using the kit of claim 12.

22. A method of amplifying nucleic acid, comprising amplifying a DNA sequence by using the composition of claim 13.

23. The method according to claim 18, wherein the improved property is selected from one or more of the following: better Mg²⁺ tolerance, better SDS tolerance, better TE tolerance, and higher long fragment amplification capability.
